(19) United States
(12) Patent Application Publication
(10) Pub. No.: US 2010/0255070 A1
(43) Pub. Date: Oct. 7, 2010

(54) COMPOSITIONS AND METHODS FOR PREVENTING, MINIMIZING AND HEALING SKIN IRRITATION AND TRAUMA

(75) Inventor: Alan E. Kligerman, Egg Harbor Township, NJ (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: PRELIEF INC., Egg Harbor Township, NJ (US)

(21) Appl. No.: 12/600,031

(22) PCT Filed: May 22, 2008
(86) PCT No.: PCT/US08/64516

§ 371 Date: Nov. 13, 2009

Related U.S. Application Data
(60) Provisional application No. 60/939,473, filed on May 22, 2007.

(51) Int. Cl. A61L 15/20; A61K 8/55; A61Q 17/00

(52) U.S. Cl. 424/445; 514/129

(57) ABSTRACT

A composition for preventing, mitigating, and alleviating irritation and skin trauma is described. The composition contains calcium glycerophosphate and at least one skin compatible, film-forming component. The composition may be applied to the skin prior to application of an adhesive tape to reduce damage to the skin which is inflicted upon removal of the tape by forming a film on the skin. Upon removal of the adhesive tape from the skin, the film, rather than skin cells, is removed with the tape. The calcium glycerophosphate component of the composition also penetrates the skin and serves to alleviate any damage which may be inflicted on the skin. The composition may also be applied to the skin to prevent subsequent irritation, such as irritation inflicted by contact with poison ivy, clothing, jewelry, insect bites, a prosthetic, etc. A medical dressing which contains the composition is also included. Finally, methods for preventing and mitigating skin irritation and trauma using the inventive composition are described.

### CROSS-REFERENCE TO RELATED APPLICATIONS

This application is a Section 371 of International Application No. PCT/US2008/064516, filed May 22, 2008, which was published in the English language on Dec. 18, 2008 under International Publication No. WO 2008/154141 A3 and which claimed priority to U.S. provisional patent application No. 60/939,473, filed on May 22, 2007, the disclosures of which are incorporated herein by reference.

### BACKGROUND OF THE INVENTION

It is well known that the skin of humans and non-human mammals is often afflicted by traumatic injuries when adhesive tapes, such as surgical tapes, adhesive bandages, nasal strips, etc., are removed from the skin. These tapes remove skin cells and thus damage and irritate the skin. Such damage is particularly inflicted when tapes are repeatedly applied and removed from the same locations on the body. It would be desirable to prevent the damage occurring from such actions and also to repair any damage which is inflicted on the skin by removing such tapes.

Several types of skin tape protectants are commercially available, such as No Sting Cavilon® Spray (commercially available from 3M), Tincture of Benzoin Pump Sprayer (commercially available from Smith & Nephew), Skin Prep Protective Dressing (Smith & Nephew), Convatec®-Allkare® Protective Barrier Wipes (commercially available from Bristol-Myers Squibb), and Skin Gel Protective Dressing Wipes (commercially available from Hollister). However, these products are designed to cling very strongly to the skin, may often be irritating, and are often so water insoluble that they cause skin damage upon attempts to remove them. If special solvents are required to remove such compositions, the solvents may themselves damage the skin. Additionally, many of these products are potentially harmful or fatal if ingested or inhaled, may cause eye irritation, and/or are flammable. Therefore, they must be used with an abundance of caution.

Many objects which are encountered in daily life also have the potential to irritate the skin. For individuals with sensitive skin or allergies, contact with latex, jewelry, and some objects of clothing (particularly those made of certain fabrics) may be especially irritating. Contact with plants such as poison ivy, poison oak, and poison sumac may produce severe itching and irritation of the skin, as may bites from some insects, such as mosquitoes and fire ants. In some cases, the irritation to the skin may be to such a dramatic extent that it results in a wound, such as from repeated scratching by the individual. A variety of different types of products may be selected by an individual for application to such irritations or wounds. For example, anti-itch preparations, such as those containing hydrocortisone, may be helpful.

Some individuals may choose to apply products which are appropriate for healing wounds to such irritations. As described in U.S. Pat. No. 7,402,323 of Applicant, compositions containing calcium glycerophosphate, preferably in combination with a pH adjusting agent comprising an α-hydroxy acid, are known to be useful for treating skin conditions by accelerating the healing of wounds or minor skin irritations. These compositions are also known to accelerate internal cellular repair in the skin.

The epidermis of humans and animals is composed of three layers: the stratum basale, the stratum spinosum and the stratum corneum, as best shown in FIG. 1. The stratum basale 2, the innermost layer, is the only layer in which active cell division occurs. When division transpires, a daughter cell, called a keratinocyte, begins to undergo terminal differentiation. By this process, the keratinocyte is sequentially transformed into a cell of the spinosum 4 and then into one of the corneum 6. Among the changes occurring during differentiation are increased synthesis of ceramide, loss of the nucleus, cell death and replacement of the cytoplasm with keratin. As keratinocytes reach the outermost stratum corneum layer 6, they are shed to the environment.

### BRIEF SUMMARY OF THE INVENTION

According to the present invention, a composition for topical application to the skin to prevent or mitigate irritation due to subsequent contact of the skin with an irritant comprises at least one skin compatible, film-forming component and calcium glycerophosphate.

A medical dressing for application to skin according to the invention contains a composition comprising calcium glycerophosphate and at least one skin compatible, film-forming component.

The invention also provides a method for preventing, alleviating, or mitigating irritation to skin occurring upon removal of an adhesive tape from the skin. The method involves applying a composition comprising calcium glycerophosphate and at least one skin compatible, film-forming component to a surface of the skin, applying an adhesive tape to the surface of the skin, and removing the adhesive tape from the skin, wherein the composition prevents, alleviates, or mitigates damage to the surface of the skin.

A method for preventing or reducing subsequent irritation to skin occurring upon contact with an external irritant comprises applying a composition comprising calcium glycerophosphate and at least one skin compatible, film-forming component to a surface of the skin to form a protected surface, and contacting the protected surface with an external irritant. The composition prevents or reduces subsequent irritation and discomfort.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a schematic diagram depicting the three layers of the human epidermis;

FIG. 2 is a diagram illustrating the inventive composition applied to the skin;

FIG. 3 is a diagram illustrating an adhesive tape applied to the composition on the skin;

FIG. 4 is a diagram illustrating removal of the tape from the skin;

FIG. 5 is a diagram illustrating the skin following removal of the tape;

FIG. 6 is a diagram illustrating the skin following removal of the residual film;

FIG. 7A is a diagram illustrating the inventive composition applied to gauze;

FIG. 7B is a diagram illustrating application of the inventive composition to gauze on the skin;

FIG. 7C is a diagram illustrating the inventive composition penetrating gauze on a wound; and

FIG. 7D is a diagram depicting a cross-section of the inventive composition in gauze on a wound.

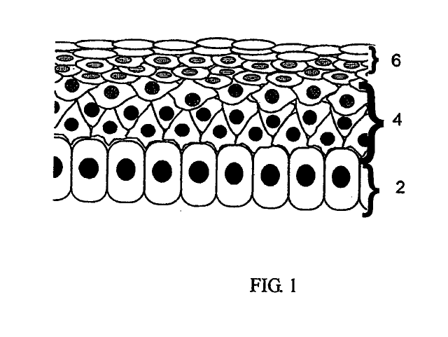

### DETAILED DESCRIPTION OF THE INVENTION

The invention is directed to compositions for topical application to the skin for preventing, alleviating and/or mitigating irritation, trauma, and damage to the skin which occurs upon removal of an adhesive or adhesive tape from the skin. As previously explained, removing traditional adhesive tapes, including bandages and surgical tapes, from the skin removes skin cells and thus damages and irritates the skin. Therefore, application of the inventive composition to the skin prior to application of an adhesive or adhesive tape, as explained below, prevents or mitigates (minimizes and corrects) such damage. The term “adhesive tape” as used herein may be understood to encompass adhesive tapes, adhesive bandages, surgical tapes, bandages, BAND-AIDS®, nasal strips, etc., as well as medical adhesives which are not attached to tapes. Any adhesive used for application to the skin, such as ostomy seals, toupee adhesives, prosthesis covers, etc., would also be included in the definition of “adhesive” or “adhesive tape.”

The compositions according to the invention comprise calcium glycerophosphate (CGP) and at least one skin compatible, film-forming component in a dermatologically acceptable carrier. The CGP and film-forming component serve distinct and different roles. Upon application to the skin, the film-forming component forms a breathable, semi-water soluble film which is deposited on the skin and adheres thereto. This ‘solubility’ is sufficient to allow removal upon gentle washing with water and soap, but not so soluble that normal non-exercise perspiration will remove it. Upon subsequent application of an adhesive or adhesive tape, the tape will adhere to the protective film, rather than to the skin surface itself, so that upon removal of the tape or adhesive, a layer of skin cells will not be removed. As described in more detail below, any residual film which remains on the skin following removal of the tape will easily wash off from the skin when desired.

In some cases, such as if the film coverage is imperfect or if the film de-adheres from the skin imperfectly, minimal damage and/or irritation to the skin may still occur upon removal of the adhesive or tape (or may be have been previously inflicted upon the skin) and the inventive composition is designed to address this damage and irritation as well. Specifically, the CGP component of the composition is skin permeable and functions as a cell repair component. That is, the CGP soaks into the skin and repairs any parts of the skin which do become irritated upon removal of the adhesive tape. Therefore, any skin damage which is not prevented by the film-forming component in the inventive compositions is mitigated by the presence of CGP. The CGP also repairs any prior damage to the skin, such as from previous applications of adhesive tapes. Such a mode of action of calcium glycerophosphate has been previously described in U.S. Pat. No. 7,402,323 of Applicant, the disclosure of which is herein incorporated by reference in its entirety.

The skin compatible, film-forming component is a water semi-soluble and alcohol insoluble, skin impermeable compound which also functions as a thickener, viscosity controller, and/or stabilizer in the compositions. Presently preferred film-forming components for use in the invention include sodium carboxymethyl cellulose, locust bean gum, microcrystalline cellulose gum, xanthan gum, and marine-derived stabilizers, such as alginates. Currently, the most preferred film-forming component is microcrystalline cellulose gum. However, the use of other known or to be developed skin compatible compounds which have similar properties is also within the scope of the invention. The film-forming component is preferably included in the compositions at a concentration of about 0.25 weight % to about 8.5 weight % based on the total weight of the composition, more preferably about 2.5 to 3.0 weight %, most preferably about 2.75 weight %. It has been found that concentrations greater than 8.5 weight % result in dry, crumbly compositions which bead up on the skin when dry and also form an undesirable and only semi-flexible, thick, shiny coating.

In a preferred embodiment, colloidal oatmeal may be included as an additional film-forming component. Colloidal oatmeal is insoluble in both aqueous and alcohol-based carriers, is known to benefit skin cells, and also contributes substance to the inventive compositions. If included, colloidal oatmeal is preferably included in the compositions at a concentration of about 0.05 to 10 weight % based on the total weight of the composition, more preferably about 1 to 3 weight %, most preferably about 2 weight %.

A second component of the compositions, calcium glycerophosphate (CGP), C₃H₇CaO₆P, is also known as 1,2,3-propanetriol, mono(dihydrogen phosphate) calcium salt (1:1), calcium glycerinophosphate, calcium phosphoglycerate and NEUROSIN®. It may exist as a hydrate, including the monohydrate and the dihydrate. Three CGP isomers exist, namely β-glycerophosphoric acid calcium salt ((HOCH₂)₂CHOPO₃Ca) and D(+) and L(−)-a-glycerophosphoric acid calcium salt (HOCH₂CH(OH)CH₂OPO₃Ca). Any one isomer, or any combination of two or more isomers, may be used as the CGP according to this invention. A commercially available form of CGP is a mixture of calcium β- and DL-α-glycerophosphates, and this is a preferred form of CGP according to the invention. The preferred form of CGP is food grade CGP according to Food Chemicals Codex (FCC) V, and may be obtained from Astha-Laboratories, Pvt. Ltd, Hyderabad, India; Seppic Inc., Fairfield, N.J., as well as Gallard Schlesinger Company, Carl Place, N.Y. 11514, which is a distributor for the Dr. Paul Lohmann GmbH KG of Emmerthal, Germany.

The preferred concentration of CGP in the inventive compositions is about 0.1 to about 50% by weight, more preferably about 4.5 to about 37 weight %, most preferably about 5.0 to about 7.5 weight %, based on the total weight of the composition.

It is known that calcium glycerophosphate has a hydrogen-ion binding capability, the means by which, in vivo and in vitro, CGP neutralizes acidic conditions on the skin when topically applied, as described in U.S. Pat. No. 5,972,321 of Applicant, which is incorporated herein by reference. This property of calcium glycerophosphate may be desirable in the inventive compositions.

The compositions preferably contain at least one buffering agent to maintain the pH of the skin at a stable, normal level and to promote normal skin flora. The preferred pH of the compositions is about 5.4 to about 7.5, more preferably about 5.6, a compromise between the average pHs of men's and women's skin, 5.45 and 5.8. Preferred buffering agents are a-hydroxy acids, such as, for example, glycolic acid, mandelic acid, malic acid, tartaric acid, citric acid, and the more preferred L-lactic acid and the D,L-lactic acid racemate. When the preferred lactic acid is used as the buffering agent, it is preferably included at a concentration of about 0.1 to about 1.9 weight %, more preferably about 1.9 weight %, to achieve the desired pH. It is also within the scope of the invention to utilize a β-hydroxy acid for pH adjustment. If an alternative buffering agent is included, the preferred concentration thereof which is necessary to achieve the desired pH may be easily determined based on the acid strength and physiological characteristics of the particular buffering agent.

Additional therapeutic components known in the art or to be developed which are appropriate for application to a wound or the immediate environs thereof may also be included in the inventive compositions. Such components include without limitation preservatives and antibiotics.

For example, the compositions preferably contain at least one pharmaceutical grade preservative, which serves as an antimicrobial agent against yeast, mold and fungi (fungistat/fungicide). It also prevents the compositions from supporting bacterial growth, which is important for protecting an aqueous product which may be stored for prolonged periods under ambient conditions. Such a component may also, to some degree, prevent bacterial growth on the skin and thus provide incidental bacteriostatic/bactericidal benefits in a wound.

The presently preferred preservative is a paraben, such as methylparaben. The parabens are known to be effective over a wide range of pH values (pH values of 4-8) and are most effective against yeast, molds and gram positive bacteria. It may be desirable to include a synergistic combination of parabens (methyl-, ethyl-, propyl- and butylparaben). The preferred concentration of paraben in the composition is about 0.02-0.4%, with a combined concentration of about 0.8% if more than one paraben is included. The paraben, for example, may also be combined with one or more known antimicrobial preservatives which are appropriate for skin care compositions.

It is also within the scope of the invention to include one or more food grade preservatives, such as natamycin or nisin, as well as various acceptable commercial antimicrobial blends. Other possible preservatives include, but are not limited to, chemical antimycotics, such as sodium benzoate and calcium propionate, as well as natural antibiotics/antiseptics, including grapefruit seed extract, tea tree oil, and olive leaf extract.

Other fungicides or fungistats, both of natural or manufactured sources, which are known in the art or to be developed for skin care (topical) compositions would also be appropriate. These types of preservative components prevent the compositions from supporting fungal or yeast growth, but are not needed as fungistats on the skin itself. Rather, the pH adjustment provided by the preferred buffering agent, which preferably brings the skin to a pH of 5.6, is believed to be sufficient to discourage the formation of molds and yeasts on the skin, and is its purpose for inclusion in the formulation.

The preferred antibiotic for inclusion in the inventive compositions is alcohol. However, the inclusion of sufficient alcohol (70% concentration) to be effective is difficult because of alcohol's tendency to coagulate calcium glycerophosphate. Therefore if alcohol of such strength is to be used, it is recommended that the alcohol be separately applied to the wound and allowed to dry prior to application of the inventive composition. It is also within the scope of the invention to include an alternative antibiotic in the composition, such as, but not limited to BETADYNE®, iodine, NEOSPORIN®, pseudomonic acid (mupirocin, commercially available as BACTROBAN®), and/or any other generic or branded wound-sanitizing product which is known in the art or to be developed.

A variety of optional ingredients may also be included in the inventive compositions. For example, a moisturizer, such as the preferred glycerin, may be desirable. Other demonstrated anti-irritants (e.g., Vaseline Intensive Care®, Aveeno®, Caladryl®, etc.), skin cell repair substances (e.g., Olay Regenerist®), antiseptics and/or wound healers (e.g., Neosporin®, Bactine®, Betadyne®), nutrients (e.g., Vitamins A, D or E, such as A & D Ointment®), and/or skin protectants (Nu-Skin®, Vaseline®)), etc. may also be included. Acids, other than those described above as buffering agents, may be utilized in appropriate concentrations and blends to adjust pH and to function as preservatives. For example, the combination of pseudomonic acid (e.g, Muciprocin® and Bactroban®) and lactic acid may be desirable in the inventive compositions because it provides bactericidal and pH adjusting (lowering) properties. It may also be desirable to include emollients, fragrances, and/or coloring agents in the compositions.

As previously explained, the compositions are preferably water based. However, the use of other vehicles is also within the scope of the invention, provided that if the compositions are applied in “wet” form (described in more detail below), the vehicle will quickly evaporate in the air so that the requisite adherable film will form. For example, an alcohol-based composition may be attractive because it may provide desirable rapid drying capabilities. The use of a dimethylsilicone or similar product as a carrier, such as DM-5 (Grant Industries, Inc., Elmwood Park, N.J. 07407), is also within the scope of the invention and may be more effective than an alcohol carrier. A preferred aqueous composition is shown in Table 1:

TABLE 1
Preferred Aqueous Composition

| | | Water | Amount |
|---|---|---|---|
| Component | Role | Solubility* | (by weight) |
| Deionized water | Carrier | S | 86.65% |
| CGP | Skin healer | LS | 7.5% |
| Microcrystalline | Film-forming component | I | 2.75% |
| cellulose gum | | | |
| Glycerin | Moisturizer | S | 1.0% |
| Methylparaben | Anti-bacterial | S | 0.2% |
| Lactic acid | Buffering agent | S | 1.9% |

*S = soluble; LS = limited solubility; I = insoluble

It can be seen that in a preferred water-based or aqueous composition, all of the components other than the film-forming component are soluble or at least limitedly soluble (absorbable into the skin). The film-forming component is designed to be insoluble so that it will form a film on the skin.

There are no limitations on the appropriate forms of the inventive compositions. Rather, it is within the scope of the invention for the compositions to be in the form of a lotion, spray, paste, free-flowing liquid, viscous liquid, cream, gel, or semi-solid, for example. Alternatively, the compositions may be provided as a powder which may be reconstituted with water into a hydrated form having any desired consistency.

The inventive compositions may be applied to the skin by any means known in the art. For example, the compositions may be applied via direct extrusion from a nozzle of a container, via spreading with hands or fingers or with a nozzle attachment, via aerosol, or from a hand-pumped spray from a tube or bottle. The compositions may be applied in a thin layer or multiple layers, but are most preferably re-applied before each adhesive or adhesive tape application.

After application to the skin, the composition will preferably dry easily in the air to form a film. The method most recommended is for the composition to be applied to the skin prior to invasion of the skin, whether at clinic or elsewhere. The desired order of events is: (1) application of bactericide, e.g., alcohol via swab; (2) application of composition; (3) utilization of local anesthetic, if such is called for; and (4) surgical procedure. If anesthetic is to be used, the time required for such to take effect will automatically create a time lapse sufficient to allow composition to dry in time for application of tape at the end of the operation. Otherwise, drying may be accelerated by artificial means, such as with a non-heat setting on a blow dryer, or by simply fanning.

The adhesive or adhesive tape may also be applied to the skin before the composition has fully dried. Application of an adhesive or adhesive tape to the composition in a more “tacky” state may deliberately down-regulate or decrease the degree of adhesion of the tape. Such decreased adhesion may be desirable in some situations. For example, a surgical incision may be held closed temporarily by moderate adhesion without the risk of pulling the incision apart when the tape is removed. Decreased adhesion may also be attractive for an athlete who is binding a limb or joint directly with tape, for example.

The compositions according to the invention are preferably water-soluble or semi-alcohol soluble and are designed to be easily washed, rinsed, or gently damp-wiped off without pulling skin cells or otherwise damaging skin at the end of the use cycle. It should be noted that the overall film is semi-water soluble, and while it will not “sweat off” or come off easily from incidental moisture, it may be easily washed off when desired. When the adhesive or adhesive tape has been ultimately removed from the skin, any film residue still clinging to the skin may then be easy removed without damaging the skin, which is an important advantage of the composition. However, the composition with the tape upon it will not be easily accidentally washed off or allowed to fall off except upon bathing or profuse perspiration by the user. In contrast, prior art “skin tape protectants”, described previously, cling very strongly to the skin, may be irritating, and are often so water insoluble that they cause skin damage upon attempts to remove them. If special solvents are required to remove such compositions, the solvents may themselves damage the skin.

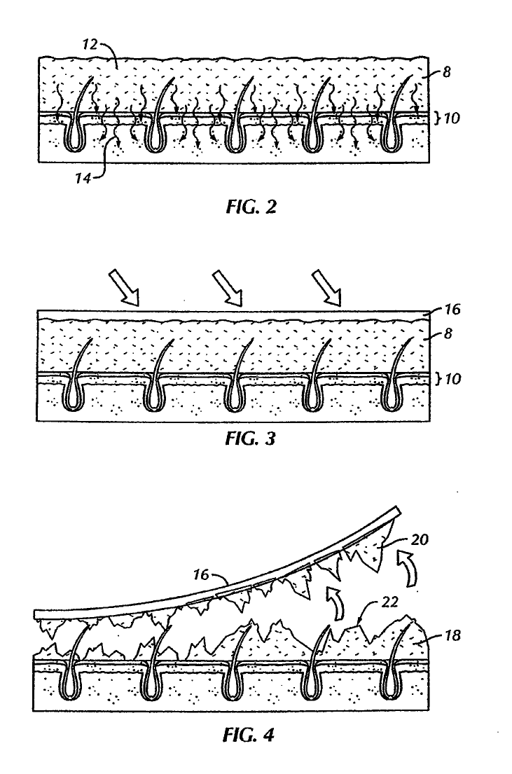

FIG. 2 depicts skin with an inventive composition applied to it. A film layer is formed on the epidermis 10. That it, it can be seen that the composition contains non-absorbable particles or fibers 12 (such as colloidal oatmeal (partly absorbable/partly non-absorbable), sodium carboxymethylcellulose gum, and other stabilizers) which remain as a self-adherent structure on the surface of the skin (film). Additionally, the composition contains absorbable (soluble) components 14, such as CGP, lactic acid, other acids, glycerin, etc., which at least partially diffuse down into the stratum corneum and stratum spinosum layers of the skin. An adhesive tape 16 may then be applied over the film layer 8, as shown in FIG. 3.

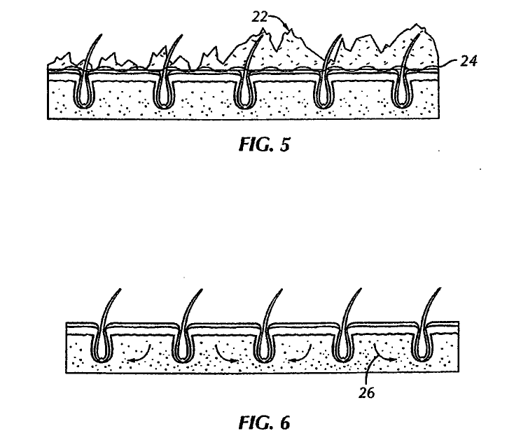

As noted above, the formation of a film on the skin surface protects the skin upon removal of a subsequently applied adhesive tape. When the tape 16 is removed, the film layer 20, rather than the skin cells, is removed, as shown in FIG. 4. The adhesion of the film to both the tape and the skin is stronger than the cohesion of the film. In other words, the composition is designed to form a protective film on the skin and to adhere essentially as strongly to skin as would adhesive tape, but to cohere slightly less strongly to itself than it adheres to skin. Thus, when adhesive tape is applied to its surface and later removed, the composition is essentially forcibly split into two film layers: a film portion 18 which remains on the skin and a film portion 20 which remains on the tape. The ideal film fracture line 22 is shown in FIGS. 4 and 5. Ideally, none or only a few skin cells will adhere to the tape itself when it is removed, since they will be protected by the remaining film layer (FIG. 5). This residual film layer may then be washed off the skin (FIG. 6), leaving cell repairing and skin growth components 26 in the skin.

However, due to imperfections in film coverage and/or selective film splitting, some skin damage may still occur upon removal of the adhesive tape. That is, as shown in FIG. 5, a lower film fracture line 24 involving loss of some skin cells may result upon removal of the adhesive tape. At this time, the composition serves a second role as a repair agent for skin cells which were not initially protected from adhesive-removal damage. The CGP-containing composition thus functions as a healing-hastening agent and pain controller to the primary wound itself or to other skin damage, as described in U.S. Pat. No. 7,402,323. As shown in FIG. 6, CGP penetrates the skin into the skin's lower layers and encourages skin renewal, thereby enabling more rapid healing of skin cells which may still be tape-damaged after the tape removal. It thus prevents, minimizes, and repairs irritation to the skin which occurs when the removal of the tape forcibly strips off some skin cells. The CGP also prevents possible irritation to sensitive skin from the adhesion of the tape that may occur while the tape is residual on the skin.

The use of the compositions according to the invention is not limited to administration prior to application of an adhesive or adhesive tape. Rather, a variety of alternative uses may be contemplated, such as a unique prophylaxis for preventing various subsequent irritations to the skin. The compositions may thus be used for topical application to the skin to prevent or mitigate irritation due to subsequent contact of the skin with an irritant. Such irritants may include, for example, poison ivy, poison oak, poison sumac, insect bites, clothing (particularly abrasive or irritating clothing), latex (particularly for latex-sensitive individuals), prosthetics, jewelry (particularly irritating jewelry), and adhesives. The presence of the film-forming component protects the skin from these irritants by forming a protective barrier film on the skin.

For example, the composition may be spread liberally over the hands, arms, or other areas to protect them from contact dermatitis caused by external irritants, such as poison ivy, poison oak, poison sumac, and the like. The composition may also be combined with standard remedies or prophylactics intended to treat such risks or conditions. Alternatively, the composition may be spread over the hands and allowed to dry prior to applying otherwise irritating clothing, such as latex gloves by persons sensitive to latex, wool or otherwise potentially irritating clothing, or jewelry. The composition may also act to protect against abrasion from prosthetics and the like. It may also be applied after removal of an adhesive to help repair or heal any skin damage which may have been inflicted by removal of the adhesive or by the adhesive itself, and thus function as a wound healant, as previously described.

The inventive compositions may also be appropriate for use in veterinary applications in which the tape-removal pulling of animals' hair is a risk, as in the binding of race horse ankles, for example, as well as other appropriate uses described above, such as when bandages are applied following surgery. A unique feature of the inventive compositions is that they coat individual hairs. That is, the compositions easily slip or split from the hairs and, as described in Example 1, have been shown to protect most hairs from forcible removal by adhesive tape removal. It is noted that in winding an adhesive tape around the damaged leg of an animal (or around an arm, finger, or leg of a human), the tape will overlap itself and at least partially adhere to itself once a first rotation around the limb or appendage has been accomplished. This permits a bandage for all intents and purposes to be as securely fashioned as if there were no pre-application film. In addition, the composition is nontoxic and thus there are no potential problems if an animal licks the area or if a child ingests some of the composition unintentionally. In contrast, prior art skin protectant compositions are typically harmful if ingested, as described previously.

The invention further relates to medical dressings for application to skin which contain a composition containing calcium glycerophosphate and at least one skin compatible, film-forming component. The term “medical dressing” is intended to include tape materials, such as bandages, gauze pads, occlusive dressings, medical adhesive tapes, surgical tapes, casts, splints, support wraps (e.g., Ace® bandages), elastic bandages, and the like, which are designed for application to skin for treatment or protection. Surgical tapes may also be used for attaching gauze, IV tubes, catheters, tubing, splints, and other medical devices to skin at home or in a hospital setting. All of these and similar products, as well as nasal strips and adhesive bandages containing such adhesive tapes, would be encompassed by the term “medical dressing.” According to the invention, at least one section of the medical dressing which is designed to come in contact with the skin contains the composition as previously described.

In one embodiment, the medical dressing may comprise an adhesive bandage having an absorptive material, a backing material having an inner surface, and an adhesive layer or portion applied to the inner surface of the backing material for treatment or protection of one area and adhering the bandage to an adjacent area of the skin. The absorptive material is located on a portion of the backing material for contact with the wound. The composition as previously described is impregnated into the absorptive material, which may further contain an antibiotic, an antiseptic, a wound healer, a nutrient, and/or a skin protectant. Appropriate backing, adhesive, and absorptive materials are well known in the art and will not be described in detail, but may be formed from the typical bandaging materials readily at hand in pharmacies and medicine cabinets.

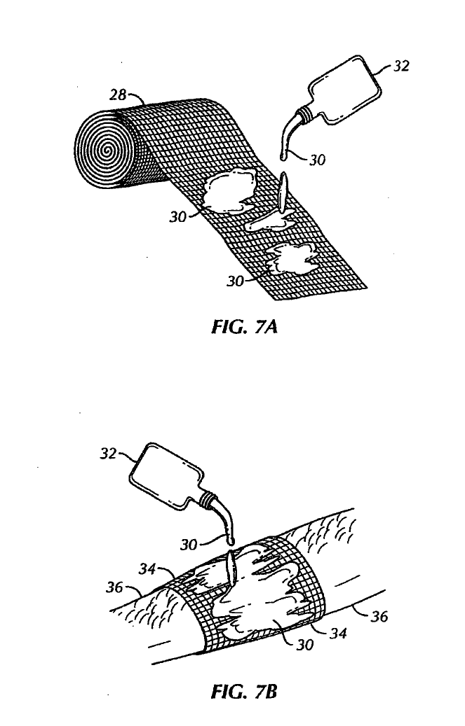

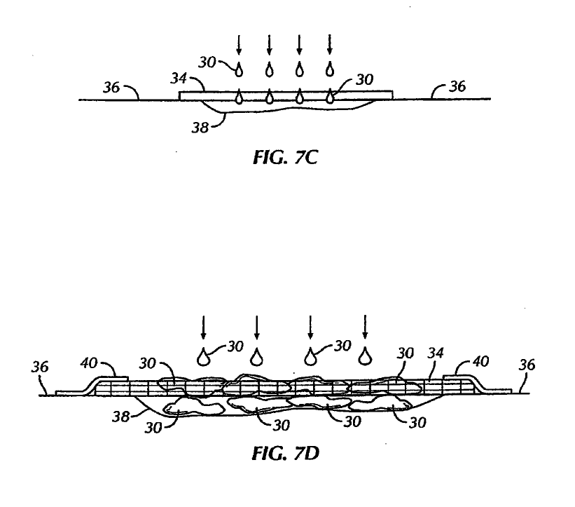

In one embodiment, the composition may be applied to the absorptive material in solution and then dried, leaving a material which is impregnated with the composition in dry form. A roll of gauze 28 to which the composition 30 is applied (such as from a container 32) prior to application to a wound is shown in FIG. 7A. The absorptive material may then be applied to the skin so that the natural moisture of the skin moistens and activates the composition, or it may be moistened by the blood from a wound or a previously applied topical antiseptic or other medication, for example. Alternatively, the composition-containing absorptive material may be moistened with water immediately prior to application to the wound. Alternatively, the composition may be spread liberally on skin and gauze may be wound around it. The gauze will self-adhere if applied while the composition is still wet. Further, outer layers of gauze 34 may optionally be spread with composition 30 (FIG. 7B) rather than to the skin 36 to further bind gauze to itself, or, more conventionally, outer layers of gauze may be fastened in place by an appropriate use of adhesive tape. As shown in FIGS. 7C-D, the composition 30 penetrates the wound 38 through the gauze 34 applied to the surface of the skin 36. As shown in FIG. 7D, tape 40 is applied on the skin 36 and over the gauze 34, to which composition 30 has been applied. The composition 30 saturates the gauze 34 and enters the wound 38.

In another embodiment, the absorptive material, particularly when part of an adhesive bandage, may be pre-moistened with a solution of the CGP-containing solution and packaged in a moisture “bubble” pancake. This pancake may be adhered to one side of the bandage and intended to be popped after application of the bandage to a wound, so that the composition floods the covered wound area. In a further embodiment, the absorptive material, particularly also when part of an adhesive bandage, is pre-moistened with a solution of the CGP-containing composition and packaged in a moisture-proof container. The moist CGP-infused absorptive material may then be applied to a wound when needed. This method is ideal when the wound is also flooded with the composition, for pain removal via pH adjustment.

The CGP-impregnated medical dressings according to the invention are attractive because they provide protective coverings for the skin with the benefits of CGP, as shown in FIGS. 7C-D. Such materials may be useful, for example, in homes, offices, and schools, in first aid kits, in hospitals and doctor's offices, and for battlefield and peacetime emergency use.

The invention also provides a method for preventing, alleviating and/or mitigating irritation and/or damage to the skin which occurs upon removal of adhesives, adhesive tapes, or similar products from the skin. As previously explained, removing adhesive tapes, including bandages and surgical tapes, from the skin removes skin cells and thus damages skin. The method involves applying a composition comprising CGP and at least one skin compatible, film-forming component to a surface of the skin, applying an adhesive or adhesive tape to the surface of the skin, and removing the adhesive tape from the surface of the skin. The composition alleviates, prevents and mitigates damage to the surface of the skin upon removal of the adhesive tape. As previously explained, the composition forms a film on the skin and it is this film which is removed with the adhesive rather than skin cells. Additionally, the CGP component penetrates the skin to heal any damage to the skin following removal of the adhesive tape.

A further method according to the invention is for preventing or reducing subsequent irritation to the skin which occurs upon contact with an external irritant, as previously described. The method involves applying the previously-described composition to a surface of the skin to form a protected surface and contacting the protected surface with an external irritant. The composition (protected surface) is now interposed between the skin and the irritant and thus prevents or reduces subsequent irritation and discomfort resulting from contact with the external irritant. The contact may be direct, such as by wearing irritating clothing or touching poison ivy, or more indirect, such as by contact with an airborne irritating particle of some sort.

This invention is further illustrated and explained by the following, non-limiting examples.

### Example 1

### Observation 1

A preliminary observation was performed to investigate the effect of applying the inventive composition to the skin on the back of the hand prior to applying two different adhesive tapes (surgical tapes and small BANDAIDS®). In this study, the composition of Table 1 was applied to the right hand of the sole subject prior to applying the adhesive tape, whereas no composition was applied to the left hand prior to applying the same adhesive tape. Each iteration (application of the composition, application of the adhesive tape, and removal of the adhesive tape) was repeated ten times and performed on two different places on each hand as follows.

A drop of the composition was squeezed from a bottle onto the appropriate location of the hand and spread with the finger tips, then allowed to dry for about 90 seconds to a satiny smoothness which allowed the fingertips to move over the skin with little friction. The appropriate adhesive tape was applied to the treated area, allowed to remain for 0.5 to 2 hours, and removed. The degree of difficulty in removal, pain to the skin upon removal, and condition of the skin were then recorded and the process was repeated. All of the used adhesive tapes were retained for subsequent observation.

In one iteration, the composition was not permitted to dry completely, but remained tacky when the adhesive tapes were applied. It was noted that the adhesive tape did not stick as aggressively to the tacky surface as to the fully dry surface. Further, the adhesive tape was easier to remove from the skin when the composition was not allowed to dry completely.

Following the study, it was found that on the untreated hand, the taped location was losing its outer cellular coverage and acquiring a smooth, dryish sheen which was not normal corneal cellular appearance. Fewer hairs remained. Removal of the tape was also becoming progressively more painful. The skin was tighter, thinner, and more tender to the touch in the footprint of the larger adhesive location and even more so on the area of the hand contacted by the adhesive segment of the BANDAID® and there were obvious hairs clinging to the removed tapes.

In contrast, on the treated hand, there was no tenderness, apparent change to the skin appearance, or increase in pain when either the adhesive tape or BANDAID® were removed. There was also no apparent build-up of the composition following ten applications on the same day to the same location of the hand. There was little or no hair clinging to the removed tapes.

The following conclusions were drawn:

(1) One portion of the composition penetrates the skin and positively affects its rate of renewal, inducing a cellular repair and/or replacement mechanism to the area of the skin it was unable to protect. Note that the rate of renewal referred to here is not obvious within the first two to three weeks, since that is the typical time required for accelerated renewal when composition is used, compared with three to four weeks or longer, for cells not so treated.

(2) A second portion of the composition remains on the skin and provides a protective film, thereby interdicting direct physical damage that surgical tape would otherwise afflict on the skin following removal.

(3) The composition does not detract significantly from the adherence of the adhesive tape to the skin but rather transfers the adherence to the protective film. There will still be a certain “skin pulling” effect when the tape is removed, but what ultimately separates from the skin is not a layer of skin cells and hair, but a split layer of the overlying film.

(4) Examination of the removed adhesive tapes revealed that the tapes which had been removed from the untreated hand had removed considerable arm hair on the first application, including complete hairs removed from their follicles. Adhesive tapes removed from the treated areas contained fewer and shorter hair fragments and no whole or complete hairs.

It will be especially noted the marked difference in presence of hairs on tape applied to untreated skin—meaning that removal of tape aggressively and painfully pulled the hairs out of the skin when the tape was removed—and the essential absence of hairs from tape removed from treated skin—meaning that removal of tape did not extract hairs, or at most, very few of them.

### Example 2

### Study 1

A clinical study was conducted in order to investigate the effects of the inventive composition shown in Table 2 on minimizing damage due to the application and removal of adhesive dressings. The study was entitled “A Randomized, Controlled, Pilot Study Assessing Topical Calcium Glycerophosphate as a Potential Agent for Minimizing Damage Due to Adhesive Dressings” (CyberDerm Laboratories, Broomall, Pa. (2006)). The purpose of the study was to determine if the inventive composition reduces redness, stratum corneum disruption, and pain/discomfort associated with the application and subsequent removal of adhesive tapes from the skin using standard criteria for assessment.

TABLE 2
Composition for Study 1

| Component | Amount (by weight) |
|---|---|
| Deionized water | 86.65% |
| CGP | 7.5% |
| Microcrystalline cellulose gum | 2.75% |
| Glycerin | 1.0% |
| Methylparaben | 0.2% |
| Lactic acid | 1.9% |
| pH of composition | 5.63 |

Each of the six 18-55 year old women in the study was initially evaluated to assess erythema (irritation), skin electrical conductivity, and skin moisture loss. Each woman had no sensitivity to cosmetics, moisturizers or adhesive dressings, no medical problems, and was not using concomitant medications that might interfere with the study results. Each panelist stopped using all moisturizing products on her back three days prior to the study.

The study was designed to assess skin condition following removal of adhesive tapes with and without prior application of the inventive composition. The assessment was performed using the following objective indicia: comparative skin moisture loss, skin electrical conductivity, and skin erythema (expert grader assessment), as well as subjective (by the patient) self-assessment of discomfort/pain upon removal of the tape.

Four 5×5 cm test sites were located on the back of each patient, two on each side. Two of these sites were treated and two were left untreated as a control, but adhesives were applied to all four sites. The product having the composition in Table 2 was applied to the appropriate sites and allowed to dry for three to five minutes. A strip of adhesive tape was then applied to and removed from each of the four sites after five minutes, after which the patient self-assessed discomfort and pain. The procedure was then repeated an additional nine times. Thirty minutes after the last tape had been removed, the following measurements were recorded: expert grader-determined erythema, chromameter a* measurement, evaporimeter measurements, and skin sensor measurements.

### Expert Grader Erythema (Irritation)

Using a relative scaling ranging from “none” to “intense,” it was found that although there appeared to be a trend for the pooled erythema scores for the treated sites to be lower than the non-treated sites following tape stripping, the trend did not prove to be statistically significant. It should be noted that erythema is not actually an indicator of skin damage, but rather a measure of blood flow stimulation as visible through the skin, even when other skin damage parameters have not been disturbed. In this case, the application and removal of tapes was sufficient to stimulate such blood flow, particularly in light of the rapid succession of such actions over a short period of time.

### Water Loss (Evaporimeter) Measurements

Water loss was determined using an evaporimeter, which measures the temperature and relative humidity at two fixed points on an axis perpendicular to the skin surface. Evaporative water loss, which assesses skin barrier function, can then be calculated.

It was found that there were dramatic differences in evaporative water loss measurements between treated and non-treated sites. Mean TEWL (transepidermal water loss) values for treated sites remained largely within the normal “uncompromised” range, whereas non-treated sites exhibited markedly elevated values (p<0.01) compared to baseline values or treated sites indicating that water loss was much higher for the untreated tape removal sites, indicating skin-sparing for the treated site.

### Chromameter a* Measurements

A Minolta Chromameter CR-200 was used to assess skin surface color using reflectance techniques. Higher a* values (indicating colors ranging from green to red) are an indication that a treatment site is more irritated. (See, for example Bubalak et al., J. Soc. Cosmet. Chem.; 37:475-479 (1986).)

It was observed that the mean chromameter a* values appeared to indicate a greater net increase in redness associated with non-treated sites. However, there did not prove to be a statistically significant difference between treated and non-treated groups.

### Skin Sensor Measurements

A DermaLab® Skin Sensor (commercially available from CyberDERM) measures current as a function of DC voltage applied to the skin surface. Using such an instrument, parameters which are related to the basic conductance properties of the stratum corneum can be derived, including onset voltage (the voltage at which current begins to rise), maximum voltage required for the current to reach a value of 2 μA, and total charge under the curve. It is believed that onset voltage is related to skin surface hydration levels and that maximum voltage and charge are measures of the barrier properties of the stratum corneum.

In this study, it was observed that mean onset voltage values after tape stripping were reduced for both treated and non-treated sites compared with baseline values. The treated sites exhibited significantly (p<0.005) higher onset values and smaller net changes from baseline than non-treated sites. Also, maximum voltage values were reduced for both treated and non-treated sites compared with baseline values. However, the treated sites exhibited significantly (p<0.005) less net change from baseline than the non-treated sites. Further, the maximum voltage values for the treated sites were higher (indicating a drier stratum corneum) compared with non-treated sites. Non-treated sites thus exhibited greater skin surface hydration-retention levels following tape stripping, likely due to removal of the drier upper layers and exposure of the moister lower skin layers. The treated site demonstrated greater skin sparing.

### Self-Assessment of Discomfort/Pain Upon Tape Removal

It does not appear that the panelists could distinguish between treated and non-treated sites in terms of a difference in discomfort or pain during any of the ten cycles in which tapes were stripped from their backs. It should be noted that the panelists were only comparing the amount of discomfort/pain and were not indicating the presence or absence of discomfort/pain upon removal of the adhesive tapes. Since the overall study demonstrated a reduction in actual skin damage on the sites treated by the composition, these “discomfort/pain” results suggest that all tapes adhered to the treated skin essentially as strongly as did tapes applied to untreated skin, and were perceived as such by the ‘blinded’ participants in terms of the “removal pull” experienced.

### Conclusions of Study

Based on this study, it was concluded that application of the inventive composition to the skin prior to application of an adhesive tape serves to reduce the physical damage caused by adhesive stripping. Both evaporative water loss and various DC voltage measurements with the Skin Sensor indicated that sites treated with the inventive composition showed significantly less compromised stratum corneum and better skin-barrier function following adhesive stripping.

### Example 3

### Study 2

A clinical study was performed in order to investigate the effects of the inventive composition shown in Table 3 on minimizing damage due to the application and removal of adhesive dressings over a five day period with a 20-24 hour dwell time. The study was entitled “A Randomized, Controlled, 5 Day Pilot Study Assessing Topical Calcium Glycerophosphate as a Potential Agent for Minimizing Skin Damage Due to Adhesive Dressings” (CyberDerm Laboratories, Broomall, Pa. (2006)). The purpose of the study was to determine if the inventive composition reduces redness, stratum corneum disruption, and pain/discomfort associated with the repeated application and subsequent removal (after 20-24 hours) of adhesive tapes from the skin during a five day period using standard criteria for assessment.

TABLE 3
Composition for Study 2

| Component | Amount (by weight) |
|---|---|
| Deionized water | 86.55% |
| CGP | 7.5% |
| Microcrystalline cellulose gum | 2.75% |
| Glycerin | 1.0% |
| Methylparaben | 0.2% |
| Lactic acid | 2.0% |
| pH of composition | 5.65 |

Each of the six 18-55 year old women in the study was initially evaluated to assess erythema (irritation), skin electrical conductivity, and skin moisture loss. Each woman had no sensitivity to cosmetics, moisturizers or adhesive dressings, no medical problems, and was not using concomitant medications that might interfere with the study results. Each panelist stopped using all moisturizing products on her back three days prior to the study.

The study was designed to assess skin condition following removal of adhesive tapes with and without prior application of the inventive composition using the objective and subjective indicia explained in Example 2.

Four 5×5 cm test sites were located on the back of each patient, two on each side. Two of these sites were treated and two were left untreated as a control, but adhesives were applied to all four sites. The product having the composition in Table 3 was applied to the appropriate sites and allowed to dry for five minutes. A strip of adhesive tape was then applied to each of the four sites, left in place for 20-24 hours, and then removed. After removal, the patient self-assessed discomfort and pain. The procedure was then repeated an additional nine times. Thirty minutes after tape removal, the following measurements were recorded: expert grader-determined erythema, chromameter a* measurement, evaporimeter measurements, skin sensor measurements, and digital photographs. The products and tape were then re-applied. This procedure (applying inventive composition, applying tape, removing tape after 20-24 hours, analysis) was repeated for a total of five days.

### Expert Grader Erythema (Irritation)

Using a relative scaling ranging from “none” to “intense,” it was found that the mean erythema scores for the treated sites were lower at every post-challenge time point compared with non-treated sites. These differences were significant (p<0.05) on days 2 and 4. For example, on day 2, thirty minutes after tape removal, the mean erythema score for the untreated site was 2.78, compared with 1.20 for the treated site. Further, on day 4, the untreated site yielded a mean score of 4.21, compared with only 3.01. These results indicate that less irritation was observed following removal of the adhesive tapes from the sites treated with the inventive composition.

### Water Loss (Evaporimeter) Measurements

Water loss was determined using an evaporimeter as previously described. It was found that mean TEWL (transepidermal water loss) values for treated sites were significantly lower (p<0.05) at every post-challenge time point during days 2, 3, 4, and 5 compared with non-treated sites. Further, the difference between the TEWL values for the treated and un-treated sites became increasing greater. By day 5, the mean TEWL for the untreated site was 41.3, relative to only 12.9 for the treated sited Also, the mean TEWL for the treated sites increased only slightly from day 1 to day 5 (from 5.7 to 12.94), whereas the mean TEWL for the untreated sites increased dramatically (from 5.73 to 41.3). These results indicate that treated sites experienced less water loss (and thus reduction in skin barrier function) following repeated tape application and removal than untreated sites.

### Chromameter a* Measurements

A Minolta Chromameter CR-200 was used to assess skin surface color as previously described. It was found that mean Chromameter a* values for the treated sites were lower (less red) at every post-challenge time point during days 2-5 compared with the non-treated sites. These differences were significant (p<0.05) on days 2 and 3. Specifically, on day 2, the treated site yielded a value of 8.63 compared with 9.70 for the untreated site, and on day 3, the treated site yielded a value of 8.95, compared with 10.62 for the untreated site. However, due to the large individual variations, these differences were not found to be statistically significant on days 4 and 5. These results indicate that skin treated with the inventive composition tended to be less red following repeated tape application and removal.

### Skin Sensor Measurements

DermaLab® Skin Sensor measurements were performed as described previously to assess skin surface hydration and barrier properties of the stratum corneum. It was found that mean onset voltage values for treated sites were significantly longer (p<0.05) at all post challenge time points during days 2-5, compared with non-treated sites, indicating a drier skin surface. This shows that for the treated sites, the skin surface barrier was protected and/or was not damaged.

It was observed that maximum voltage values for the treated sites were higher (indicating a drier stratum corneum) at all post-challenge time points compared with non-treated sites. These differences were significant (p<0.01) on days 3-5. For example, on day 3, the treated site exhibited a value of 27.08, compared with 14.99 for the untreated site.

Finally, it was found that the charge values for the treated sites were greater (indicating less stratum corneum disruption) at all post challenge time points during days 2-5 than the non-treated sites. These differences were significant (p<0.05) on days 4 and 5. For example, on day 4, the treated site had a value of 1.79, compared with 1.18 for the untreated site.

### Self-Assessment of Discomfort/Pain Upon Tape Removal

It was found that mean panelist ratings of discomfort favored the treated sites at all post challenge time points compared with the non-treated sites. Although the difference was only statistically significant on day 5, there also appeared to be a tendency favoring the treated sites on days 3 and 4.

Based on this study, it was concluded that assessments of erythema and Chromameter measurements of redness revealed that the sites treated with the inventive composition were associated with less redness than non-treated sites due to adhesive stripping. Instrumental measures of both evaporative water loss and electrical properties of the skin also indicated that sites treated with the inventive composition exhibited significantly less disruption of the stratum corneum barrier than non-treated sites. In addition, there were indications from panelist self-assessments that removal of the adhesive tapes from treated sites resulted in less discomfort than from non-treated sites.

### Example 4

### Study 3

A clinical study was conducted in order to further investigate the effects of three different inventive compositions, shown in Table 4, on minimizing damage due to the application and removal of adhesive dressings from the skin. The study was entitled “A Randomized, Controlled, Pilot Study Comparing Potential Agents for Minimizing Damage Due to Adhesive Dressings,” (Cyberderm Laboratories, Broomall, Pa. (2006)). The purpose of the study was to determine how the inventive compositions reduce redness and stratum corneum disruption associated with the application and subsequent removal of adhesive tapes from the skin using standard criteria for assessment.

TABLE 4
Compositions for Study 3

| | | Amount | |
|---|---|---|---|
| Component | Composition A | (by weight) | Composition A |
| Deionized water | 86.45% | Deionized water | 86.45% |
| CGP | 7.5% | CGP | 7.5% |
| Microcrystalline | 2.75% | Microcrystalline | 2.75% |
| cellulose gum | | cellulose gum | |
| Glycerin | 1.0% | Glycerin | 1.0% |
| Methylparaben | 0.2% | Methylparaben | 0.2% |
| Lactic acid | 2.1% | Lactic acid | 2.1% |
| Gransil DM-5 | — | Gransil DM-5 | — |
| Silicone gel | | Silicone gel | |
| Colloidal Oatmeal | — | Colloidal Oatmeal | — |
| pH of Composition | 5.64 | pH of | 5.64 |
| | | Composition | |

Each of the six 18-55 year old women in the study was initially evaluated to assess erythema (irritation), skin electrical conductivity, and skin moisture loss. Each woman had no sensitivity to cosmetics, moisturizers or adhesive dressings, no medical problems, and was not using concomitant medications that might interfere with the study results. Each panelist stopped using all moisturizing products on her back three days prior to the study.

The study was designed to assess skin condition following removal of adhesive tapes with and without prior application of the inventive compositions. The assessment was performed using the following objective indicia: comparative skin moisture loss, skin electrical conductivity, and skin erythema (expert grader assessment).

Four 5×5 cm test sites were located on the back of each patient, two on each side. Three of these sites were treated with different compositions (A, B, and C), and one was left untreated as a control, but adhesives were applied to all four sites. The products having the compositions in Table 5 were applied to the appropriate sites and allowed to dry for four minutes. A strip of adhesive tape was then applied to and removed from each of the four sites after five minutes. The procedure was then repeated an additional nine times. Thirty minutes after the last tape had been removed, the following measurements were recorded: expert grader-determined erythema, chromameter a* measurement, evaporimeter measurements, and skin sensor measurements. Digital photographs were taken 30-40 minutes following completion of the ten cycles.

### Expert Grader Erythema (Irritation)

Using a relative scaling ranging from “none” to “intense,” it was found that the trend for the pooled erythema scores for the “C” treated sites appeared to be slightly greater and that of the “A” and “B” treated sites to be slightly less than the non-treated sites following tape stripping. The trend did not prove to be statistically significant.

### Water Loss (Evaporimeter) Measurements

Water loss was determined using an evaporimeter, as previously described. It was found that there were dramatic differences in evaporative water loss measurements between treated and non-treated sites. Mean TEWL (transepidermal water loss) values for treated sites remained largely within the normal “uncompromised” range, whereas non-treated sites exhibited markedly elevated values compared to baseline values or treated sites.

### Chromameter a* Measurements

A Minolta Chromameter CR-200 was used to assess skin surface color as previously described. The results indicate an increase in redness in all test groups thirty minutes after completion of the ten tape stripping cycles. Also, sites which were treated with compositions “A” and “B” were associated with less redness than untreated sites.

### Skin Sensor Measurements

Using a DermaLab® Skin Sensor, as previously described, it was found that mean onset voltage values after tape stripping were reduced for both treated and non-treated sites compared with baseline values. All treated sites exhibited higher onset values and smaller net change from baseline than non-treated sites, indicating that non-treated sites exhibit greater skin surface hydration levels following tape stripping, likely due to removal of the drier upper layers and exposure of the moister lower skin layers.

Post-stripping values for maximum voltage were reduced for both treated and non-treated sites relative to baseline values, indicating greater disruption of the stratum corneum barrier. Finally, it was observed that the total charge values were reduced for both treated and non-treated sites compared to baseline values. However, there was no significant difference in the net change from the baseline measurement between the treated and non-treated groups.

### Conclusions of Study

Based on this study, it was concluded that application of the inventive compositions to the skin prior to application of an adhesive tape serves to reduce the damage caused by adhesive stripping. Evaporative water loss measurements indicated that sites treated with the inventive compositions showed significantly less compromised stratum corneum and better skin barrier function following adhesive stripping. Various DC voltage measurements with the Skin Sensor suggested that the inventive compositions provided a protective influence.

It is of interest that in the multiple same-day tape strippings, that there was no correlation between the extent of redness (erythema) and the assessed disruption to the stratum corneum as measured by evaporative water loss, whereas in the longer term tape strippings there was such correlation. In the case of the short term strippings, it is assumed that it was the sheer amount of concentrated activity that stimulated blood flow in both treated and untreated sites. Since there was redness (erythema) only in the untreated sites over the longer term tape stripping study, during which time there was no such concentration of skin activity, it becomes even clearer that the use of the inventive composition does protect the skin from damage, even as perceived by the body, itself.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A composition for topical application to skin to prevent or mitigate irritation due to subsequent contact of the skin with an irritant, wherein the composition comprises at least one skin compatible, film-forming component and calcium glycerophosphate.

2. The composition according to claim 1, further comprising at least one buffering agent.

3. The composition according to claim 1, further comprising at least one component selected from the group consisting of a preservative and an antibiotic.

4. The composition according to claim 1, further comprising at least one component selected from the group consisting of colloidal oatmeal, a moisturizer, a cell-growth substance, and an anti-irritant.

5. The composition according to claim 1, wherein the skin compatible, film-forming component is selected from the group consisting of sodium carboxymethylcellulose, locust bean gum, microcrystalline cellulose gum, xanthan gum, and a marine-derived stabilizer.

6. The composition according to claim 2, wherein the buffering agent comprises an α-hydroxy acid.

7. The composition according to claim 2, wherein the buffering agent comprises lactic acid and the film-forming component comprises microcrystalline cellulose.

8. The composition according to claim 7, further comprising at least one component selected from the group consisting of a moisturizer and a preservative.

9. The composition according to claim 8, wherein the moisturizer comprises glycerin and the preservative comprises methylparaben.

10. The composition according to claim 1, wherein the irritant is selected from the group consisting of poison ivy, an insect bite, poison oak, poison sumac, clothing, latex, a prosthetic, jewelry, and an adhesive.

11. The composition according to claim 1, wherein the composition is in a form of a lotion, spray, paste, liquid, cream, gel, a semi-solid, or a powder.

12. A medical dressing for application to skin, wherein the medical dressing contains a composition comprising calcium glycerophosphate and at least one skin compatible, film-forming component.

13. The medical dressing according to claim 12, wherein the dressing comprises an absorptive material and the composition is impregnated into the absorptive material.

14. The medical dressing according to claim 12, wherein the composition further comprises at least one buffering agent.

15. The medical dressing according to claim 13, wherein the absorptive material further contains at least one component selected from the group consisting of an antibiotic, an antiseptic, a wound healer, a nutrient, and a skin protectant, and mixtures thereof.

16. The medical dressing according to claim 12, comprising an adhesive bandage having an absorptive material, a backing material having an inner surface, and an adhesive portion on the inner surface of the backing material, wherein the composition is present in the absorptive material.

17. A method for preventing, alleviating, or mitigating irritation to skin occurring upon removal of an adhesive tape from the skin, the method comprising applying a composition comprising calcium glycerophosphate and at least one skin compatible, film-forming component to a surface of the skin, applying an adhesive tape to the surface of the skin, and removing the adhesive tape from the skin, wherein the composition prevents or mitigates damage to the surface of the skin.

18. The method according to claim 17, wherein the adhesive tape is selected from the group consisting of surgical tape, a medical adhesive, a bandage, an adhesive bandage, and a nasal strip.

19. The method according to claim 17, wherein the composition further comprises at least one buffering agent.

20. The method according to claim 17, wherein the composition is formulated to form a protective film on the skin and to simultaneously allow components of the composition to penetrate into the skin.

21. A method for preventing or reducing subsequent irritation to skin occurring upon contact with an external irritant, the method comprising applying a composition comprising calcium glycerophosphate and at least one skin compatible, film-forming component to a surface of the skin to form a protected surface and contacting the protected surface with an irritant, wherein the composition prevents or reduces subsequent irritation and discomfort.

22. The method according to claim 21, wherein the irritant is selected from the group consisting of poison ivy, an insect bite, poison oak, poison sumac, clothing, jewelry, latex, an adhesive, and a prosthetic.
